(19) United States
(12) Patent Application Publication
(10) Pub. No.: US 2012/0220787 A1
(43) Pub. Date: Aug. 30, 2012

(54) PROCESS FOR THE PREPARATION OF 5-/6-NITROFLUORESCEIN

(76) Inventors: Stuart Wharton, Chester (GB); Tomos Huw Roberts, Bangor (GB)

(21) Appl. No.: 13/201,372

(22) PCT Filed: Feb. 10, 2010
(86) PCT No.: PCT/GB2010/050206

§ 371 Date: Apr. 13, 2012

(30) Foreign Application Priority Data
Feb. 12, 2009 (GB) 0902286.4

(51) Int. Cl. C07D 493/10

(52) U.S. Cl. 549/223

(57) ABSTRACT

A process for the preparation of a mixture of 3′,6′-dihydroxy-6-nitrospiro[2-benzofuran-3,9′-xanthene]-1-one and 3′,6′-dihydroxy-5-nitrospiro[2-benzofuran-3,9′- xanthene]-1-one comprising the steps of:- (a) reacting 4-nitrophthalic acid or 4-nitrophthalic anhydride with benzene-1,3-diol in methanesulphonic acid; (b) quenching the reaction in step (a) with a solvent to precipitate product; (c) isolating the precipitate; (d) heating the precipitate in water in order to hydrolyse any methansulphonic acid ester present.

The present invention relates to an improved process for the preparation of 5-/6 nitrofluorescein.

5-Nitrofluorescein (3′,6′-dihydroxy-6-nitrospiro[2-benzofuran-3,9′-xanthene]-1-one) is a key intermediate in the synthesis of various fluorescent compounds such as 5-aminofluorescein (5-amino-3′,6′-dihydroxy-3H-spiro[isobenzofuran-1,9′-xanthen]-3-one), fluorescein 5-isothiocyanate (FITC; 3′,6′-dihydroxy-5-isothiocyanato-3H-spiro[isobenzofuran-1,9′-xanthen]-3-one), and fluorescein lisicol (previously known as cholyl-lysylfluorescein or CLF; N6-({3′,6′-dihydroxy-3-oxospiro[isobenzofuran-1(3H),9′-xanthen]-5-yl}thiocarbamoyl)-N2-(3 ,7,12-trihydroxy-5-cholan-24-oyl)-L-lysine). Fluorescein 5-isothiocyanate has a wide range of biological applications including use as a fluorescent labeling agent for proteins, as a fluorescent reagent for protein tracing, and as a reagent in a fluorescent antibody technique for the rapid identification of pathogens. In addition, the use of fluorescent bile acid derivatives, and CLF in particular, in a method for the determination of the liver function of a human or animal subject is described in EP1,003,458 (Norgine Europe BV).

A. H. Coons and M. H. Kaplan (Journal of Experimental Medicine 1950, 91, 1-13) describe the synthesis of 5-/6-nitrofluorescein by the thermal condensation of a dry mixture of 4-nitrophthalic acid and resorcinol at 195-200° C. for 12 to 18 hours to give a 98% yield of crude 5-/6-nitrofluorescein. No indication is given in the paper regarding the isomer ratio or the degree of conversion. However, in the subsequent acetylation and separation of isomers, Coons and Kaplan report a 26% yield of 5-nitrofluorescein diacetate.

Importantly, the reaction mixture becomes a solid mass during the thermal condensation reaction and recovery of the product proves to be extremely difficult when the reaction is scaled up. Coons and Kaplan report the reaction on a 100 gram scale, i.e. 100 g 4-nitrophthalic acid and 100 g resorcinol as starting materials, and describe the solidified melt being chipped from the beaker in which the reaction was conducted. However, when the scale of the reaction is increased, and when the reaction is conducted under an inert atmosphere, it has been our experience that recovery inevitably requires destruction of the reaction vessel in order that the solid product can be chipped out prior to further processing. This becomes uneconomic when the reaction is scaled up to produce kilo quantities of the intermediate.

Furthermore, and importantly, we have found that there is a serious health and safety concern on scale up due to the risk of explosion. In our investigations, an uncontrollable and violent exothermic reaction can occur when the reaction is carried out on above a 50 g-100 g scale.

In summary, the prior art process is unworkable on scale up and there is a long-felt need in the industry to provide a process which can be used safely to produce kilo or multi-kilo batches of 5-/6-nitrofluorescein and which does not require protracted reaction times or elaborate work up procedures.

A main object of the present invention is to provide a high yielding process for the preparation of 3′,6′-dihydroxy-6-nitrospiro[2-benzofuran-3,9′-xanthene]-1-one and 3′,6′-dihydroxy-5-nitrospiro[2-benzofuran-3,9′-xanthene]-1-one.

Another object of the present invention is to provide a process for the preparation of 3′,6′-dihydroxy-6-nitrospiro[2-benzofuran-3,9′-xanthene]-1-one and 3′,6′-dihydroxy-5-nitrospiro[2-benzofuran-3,9′-xanthene]-1-one with relatively short reaction times.

Yet another object of the present invention is to provide such a process to prepare a mixture of 3′,6′-dihydroxy-6-nitrospiro[2-benzofuran-3,9′-xanthene]-1-one and 3′,6′-dihydroxy-5-nitrospiro[2-benzofuran-3,9′-xanthene]-1-one in substantially pure form without elaborate work up procedures. The term “substantially pure” refers to material that is not less than 85% pure. A more preferred purity for the product derived from the present process is not less than 89% pure and typically the product so produced has a purity of about 89 to 94%. Purity can be determined by a number of conventional analytical methods, including HPLC and n.m.r. analyses.

The present invention may be described by the following reaction scheme:

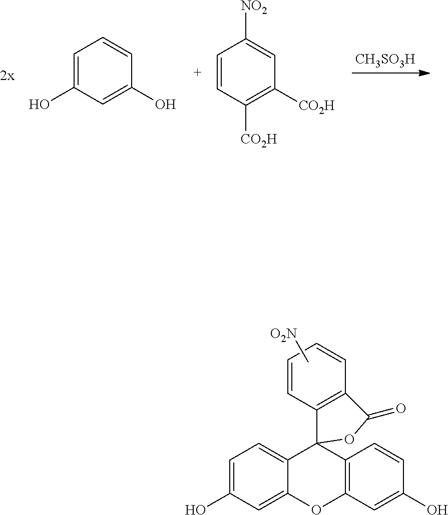

Accordingly, there is provided a process for the preparation of a mixture of 3′,6′-dihydroxy-6-nitrospiro[2-benzofuran-3,9′-xanthene]-1-one and 3′,6′-dihydroxy-5-nitrospiro[2-benzofuran-3,9′-xanthene]-1-one comprising reacting 4-nitrophthalic acid or 4-nitrophthalic anhydride with benzene-1,3-diol in methanesulphonic acid comprising the steps of:

(a) reacting 4-nitrophthalic acid or 4-nitrophthalic anhydride with benzene-1,3-diol in methanesulphonic acid;

(b) quenching the reaction in step (a) with a solvent to precipitate product;

(c) isolating the precipitate;

(d) heating the precipitate in water in order to hydrolyse any methansulphonic acid ester present.

It should be noted that 4-nitrophthalic anhydride can be used interchangeably with 4-nitrophthalic acid as a starting material in this condensation reaction. Indeed, mixtures of 4-nitrophthalic acid and 4-nitrophthalic anhydride could be used as starting materials if required.

Carrying out the condensation reaction in methanesulphonic acid instead of by thermal fusion provides much milder and safer reaction conditions over a very much shorter reaction time.

Any potential exotherm during the condensation reaction can be controlled by controlling the reaction temperature, optionally by heating with a water bath.

Preferably the reaction is carried out at a temperature in the range of from about 60° to about 120° C., more preferably above about 90° C. A particularly preferred temperature range for carrying out the condensation reaction is about 95° to 100° C.

Preferably the methanesulphonic acid is substantially pure. More preferably the methanesulphonic acid is not less than 95% w/w. In a particularly preferred embodiment the methanesulphonic acid is not less than 98% w/w.

Preferably, the reaction environment is selected such that the product is precipitated from the reaction mixture. Accordingly, the reaction mixture is more preferably quenched with a solvent and more preferably quenched to precipitate the product from the reaction mixture. In a particularly preferred embodiment the solvent used comprises an aqueous medium. In a further preferred embodiment the reaction mixture is added to water to precipitate the product. The final step of the process is one that will enable the product to be isolated from the reaction mixture and is preferably filtration. However, the product may also be obtained by centrifugation of the reaction mixture after precipitation of the product.

When water is used as a solvent to quench the reaction, the temperature of the water prior to quenching the reaction mixture is preferably 0° to 10° C., and preferably the temperature of the aqueous phase is maintained at 30° C. or less during the addition process.

Preferably the ratio of water to methanesulphonic acid in the quench is about 4.5 w/w or less, and more preferably about 3 w/w or less.

In a particularly preferred embodiment any methanesulphonic acid ester present in the product is hydrolysed. Therefore, especially preferred is when the precipitated product is preferably heated in water at 80° to 100° C., more preferably with agitation.

The aqueous hydrolysis step is preferably repeated until the amount of residual methanesulphonic acid ester is below a pre-determined level. A suitable pre-determined level for the amount of residual methanesulphonic acid ester is about 5% or less. The product from this hydrolysis step may be isolated by filtration or centrifugation.

Preferably the isolated product is dried in vacuum at up to about 65° to 70° C.

In the condensation reaction, the volume of methanesulphonic acid in the reactants, based on the weight of 4-nitrophthalic acid or 4-nitrophthalic anhydride, is preferably about 2 to 10 v/w and more preferably about 3 to 5 v/w.

In order to achieve the formation of the desired mixed nitrofluorescein isomers, a reactor containing 4-nitrophthalic acid and resorcinol (benzene-1,3-diol) in methanesulphonic acid is heated, preferably to over 90° C. and more preferably 95-100° C. Failure to maintain the temperature or reaction time may result in incomplete reaction.

During the synthesis the end point of the reaction (determined as the time when 4-nitrophthalic acid HPLC area is preferably less than 2% area) is determined by in-process analysis of the reaction mixture. This limit is important in obtaining intermediate product that meets specification.

Suitable HPLC conditions include using a Waters (RTM) XBridgeShield RP18 column 100 mm in length, 4.6 mm in diameter with a particle size of 3.5 μm. The mobile phase may be a water containing 0.1% H₃PO₄ and/or acetonitrile containing 0.1% H₃PO₄ with a mixing ratio of between 100% aqueous phase and 100% acetonitrile phase over 25 minutes.

Once the reaction is complete it is allowed to cool and may be quenched by pouring into a solvent, for example water. The resulting solid is filtered and transferred back to the reaction vessel and water is added and the mixture hydrolyzed, preferably at 95-100° C., and preferably for at least 30 minutes. On cooling the solid is isolated, preferably by filtration or centrifugation, recharged to the reaction vessel and hydrolysed with a further portion of water at, again preferably at 95-100° C. for 30 minutes. Failure to maintain the optimum temperature and time for the hydrolysis may result in the incomplete hydrolysis of any methanesulphonic acid ester present.

The reaction is again allowed to cool and the solid isolated by filtration or centrifugation and dried to constant weight in a vacuum oven at 65-70° C. Failure to maintain the temperature may result in decomposition of the product in the event of overheating.

In summary, what appeared to be methanesulphonic acid contamination of the crude condensation product was found to be due to the formation of methanesulphonic acid ester of one or both phenol groups. Although this esterfied product is substantially insoluble in water it has unexpectedly and counter-intuitively been found that complete hydrolysis of any methanesulphonic acid ester can be achieved simply by heating or refluxing the precipitate from the cold water quench in water and repeating this aqueous hydrolysis if necessary. Aqueous hydrolysis avoids digestion with hydrochloric acid, which is a requirement after the thermal condensation process A. H. Coons and M. H. Kaplan (Journal of Experimental Medicine 1950, 91, 1-13).

The isomer ratio of 5-/6-nitrofluorescein produced in the methanesulphonic acid catalysed condensation is in the order of about 60:40 to about 70:30 5-nitrofluorescein:6-nitrofluorescein.

A mixture of 5-/6-nitrofluorescein prepared according to the present invention has a number of applications. For example, as referred to above, it can be used to synthesise fluorescein 5-isothiocyanate according to reaction Scheme 1 below:

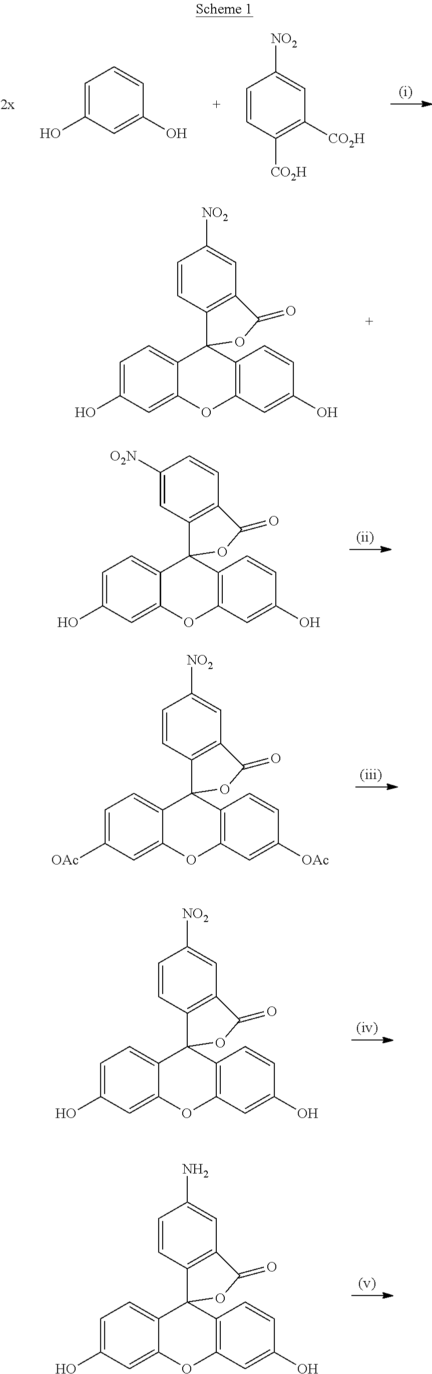

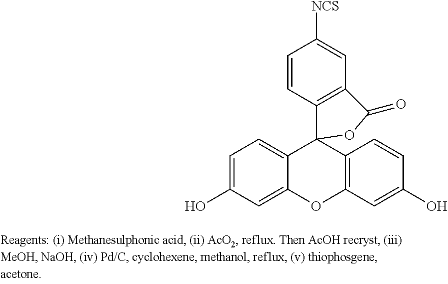

Fluorescein 5-isothiocyanage (FITC), and thus 5-/6 nitrofluorescein, can be used to synthesise fluorescein lisicol (previously known as cholyl-lysylfluorescein (CLF))according to Scheme 2 shown below.

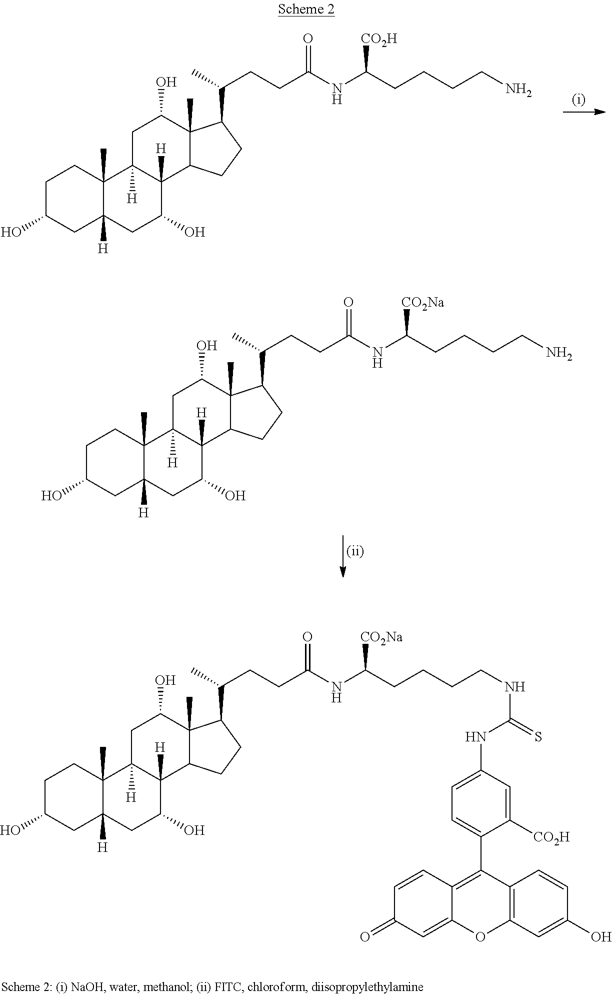

### EXAMPLE 1

### Preparation of 5-/6-nitrofluorescein

### Reactor 1

A reactor previously made inert with nitrogen was charged with methanesulphonic acid (2.43 L). 4-nitrophthalic acid (600 g) was added, and the temperature maintained between 20-25° C. Following agitation for 10 minutes, resorcinol (benzene-1,3-diol) (657 g) was added and the reaction mixture heated cautiously to 70° C. then to 95-100° C. The reaction mixture was stirred at 95-100° C. and stirring and heating was continued for a minimum of 2 hours. The reaction mixture was sampled every 2 hours for in-process analysis. Following reaction completion, indicated by a limit of not greater than 2% 4-nitrophthalic acid by HPLC, the reaction was cooled to 60-70° C.

### Reactor 2

A second reactor was charged with cold (0-10° C.), deionised water (7.2 L). The reaction mixture at 60-70° C. was charged to the cold water, keeping the temperature of the solution at less than 30° C. The mixture was agitated for a minimum of 30 minutes and the precipitated product isolated by filtration. The isolated material was washed with deionised water (0.36 L) and pulled dry under vacuum.

The damp product was recharged to the reactor, deionised water (4.8 L) added and the mixture heated to 95-100° C. with agitation for 30 minutes. The suspension was cooled to 20-30° C. and the solid product isolated by filtration, washed with deionised water (0.48 L) and pulled dry. The damp product was analysed by proton NMR to determine the methanesulphonic acid ester content. A methanesulphonic acid ester content of less than 5% relative to the product is preferred. The hydrolysis procedure was repeated until the ester content was within this limit. The solid was transferred to a vacuum oven and dried at 65-70° C. until constant weight to give the product as a dark orange solid.

m.p =346-349° C.

¹H (DMSO-D₆) 6.54-6.58 (6H, m, 6-isomer) 6.58-6.72 (6H, m, 5-isomer), 7.57 (1H, d, 5-isomer), 8.1 (1H, d, 6-isomer), 8.26 (1H, d, 6-isomer), 8.5 (1H, dd, 6-isomer), 8.57 (1H, dd, 5-isomer), 8.66 (1H, d, 5-isomer), (1H, m), 4.15 (1H, d)

m/z=378.1 (M+1)

In the above example, the precipitated product was isolated from the reaction mixture by filtration. It will be understood that isolation of the precipitated product may also be achieved by centrifugation.

1. A process for the preparation of a mixture of 3′,6′-dihydroxy-6-nitrospiro[2-benzofuran-3,9′-xanthene]-1-one and 3′,6′-dihydroxy-5-nitrospiro[2-benzofuran-3,9′-xanthene]-1-one comprising the steps of:
(a) reacting 4-nitrophthalic acid or 4-nitrophthalic anhydride with benzene-1,3-diol in methanesulphonic acid;
(b) quenching the reaction in step (a) with a solvent to precipitate product;
(c) isolating the precipitate;
(d) heating the precipitate in water in order to hydrolyse any methansulphonic acid ester present.

2. The process as claimed in claim 1 wherein the reaction in step (a) is carried out at about 60° to 120° C.

3. The process as claimed in claim 2 wherein the reaction in step (a) is carried out at above about 90° C.

4. The process as claimed in claim 3 wherein the reaction in step (a) is carried out at about 95° C. to 100° C.

5. The process as claimed in any one of claims 1 to 4 inclusive wherein the methanesulphonic acid is not less than 95% w/w.

6. The process as claimed in claim 5 wherein the methanesulphonic acid is not less than 98% w/w.

7. The process as claimed in claim 1 wherein the reaction in step (a) is quenched by adding the reaction to the solvent.

8. The process according to claim 7 wherein the reaction in step (a) is quenched with water to precipitate the product.

9. The process according to claim 8 wherein the ratio of water to methanesulphonic acid is about 4.5 w/w or less.

10. The process according to claim 9 wherein the ratio of water to methanesulphonic acid is about 3 w/w or less.

11. The process as claimed in claim 9 wherein the temperature of the water before the reaction is quenched is 0° to 10° C.

12. The process as claimed in any one of claims 9 to 11 inclusive wherein the temperature of the water is maintained at 30° C. or less during the period in which the reaction is quenched.

13. The process as claimed in claim 1 wherein the precipitated product in step (c) is isolated by filtration.

14. The process as claimed in claim 1 inclusive wherein the precipitated product in step (c) is isolated by centrifugation.

15. The process as claimed in claim 1 wherein the precipitate in step (d) is heated in water at about 80° to 100° C.

16. The process as claimed in claim 1 wherein the precipitate in step (d) is heated with agitation.

17. The process according to claim 1 wherein the precipitate from step (d) is isolated and wherein the step of heating the precipitate in water is repeated.

18. The process according to claim 17 wherein the step of heating the precipitate in water is repeated until the level of methanesulponic acid ester present is about 5% or less.

19. The process as claimed in claim 1 wherein the product from step (d) is subjected to drying in a vacuum.

20. The process as claimed in claim 19 wherein the product is dried at between room temperature and 70° C.

21. The process as claimed in claim 1 wherein the ratio of methanesulphonic acid to 4-nitrophthalic acid/4-nitrophthalic anhydride in step (a) is about 2 to 10 v/w.

22. The process as claimed in claim 21 wherein the ratio of methanesulphonic acid to 4-nitrophthalic acid/4-nitrophthalic anhydride is about 3 to 5 v/w.

23. (canceled)
